(19) United States
(12) Patent Application Publication
(10) Pub. No.: US 2005/0019757 A1
(43) Pub. Date: Jan. 27, 2005

(54) Contaminant detection apparatus

(76) Inventor: Danylo J. Stolarchuk, Etobicoke, CA (US)

Correspondence Address: VIDAS, ARRETT & STEINKRAUS, P.A., 6109 BLUE CIRCLE DRIVE, SUITE 2000, MINNETONKA, MN, 55343-9185, US

(21) Appl. No.: 10/868,341

(22) Filed: Jun. 14, 2004

Related U.S. Application Data
(60) Provisional application No. 60/477,778, filed on Jun. 12, 2003.

(51) Int. Cl. C12Q 1/70; C12Q 1/68

(52) U.S. Cl. 435/5; 435/6; 435/287.2

(57) ABSTRACT

A contaminant detection apparatus comprising a powered portable detection device for detecting the presence of at least one pathogen in a sample; a test applicator kit comprising a sample applicator and a cartridge configured to receive and retain the sample applicator; the sample applicator including a swab having a first and second swab heads for swabbing a surface to obtain a sample, the cartridge and sample applicator being configured such that when the sample applicator is retained in the cartridge, the first swab head is retained in said preserving chamber to preserve a confirmatory version of said sample, and the second swab head is positioned in the solvent chamber to dissolve the second swab head to a substantially liquid mixture including said sample, and permit the mixture to flow via flow paths to the wells.

### FIELD OF THE INVENTION

This invention relates to the field of pathogen/contaminant detection, and more particularly, to the field of devices for pathogen detection.

### BACKGROUND OF THE INVENTION

Pathogens, such as viruses, bacteria, toxins and other contaminants, are omnipresent. Under particular circumstances, such pathogens can become dangerous to human health. For example, the presence of salmonella or E coli in food can be injurious or fatal to those consuming the food. Anthrax spores can be injurious or fatal to a person who touches or inhales them. There are many examples of circumstances in which particular pathogens can be dangerous to people.

Apart from the direct danger to humans, there is a serious and detrimental impact associated with indeterminate or tentative discoveries of pathogens. For example, if a suspicion arises that certain food is carrying a pathogen, then, typically, samples would be taken and transported to a lab, where tests would be conducted on the samples under carefully controlled environmental conditions. Until the test results are finally determined, the food (or other environment where the pathogen is suspected) is held in limbo. This delay can be very costly.

In addition to the cost of delay, there are serious costs associated with incorrect test results. If a test incorrectly returns a negative result, injury or death to humans may result. If a test incorrectly returns a positive result, then food may be destroyed, premises closed, equipment discarded, or disinfection procedures initiated all because of an incorrect test result.

Tests for pathogens are typically conducted by mixing the sample with a mix of pathogen-specific extraction chemicals, so that if the pathogen is present, light is emitted. This light emission is typically of low intensity, and a Photo Multiplier Tube (PMT) is typically used to sense the emission. A typical PMT consists of seven photosensitive plates arranged in series, with each plate having a successively higher potential applied thereto. The potential difference between each successive plate is typically 100 volts. The first plate releases electrons in response to light, and these electrons are drawn to the next plate because of its higher potential. The third plate draws still more electrons from the second plate because of its still higher potential, and so on. In typical PMTs, the total gain can be adjusted by adjusting the voltage applied to the PMT. For example, a typical PMT may be adjusted so that the potential difference between successive plates is 110 volts instead of 100 volts. The PMT emits an amplified signal which indicates whether light was emitted by the sample. However, PMTs are sometimes imprecise and unreliable.

### SUMMARY OF THE INVENTION

Therefore, what is preferred in one aspect is a contaminant detection apparatus effective and convenient for use in the field. What is desired in another aspect is an optical sensor module that improves precision and reliability.

Therefore, in one aspect, there is provided a contaminant detection apparatus comprising:
a powered portable detection device for detecting the presence of at least one pathogen in a sample;
a test applicator kit comprising a sample applicator and a cartridge configured to receive and retain the sample applicator. The cartridge preferably includes a solvent chamber for holding a solvent, preferably includes a preserving chamber for holding a preserver, and preferably includes at least one well for holding at least one pathogen-specific set of extraction chemicals. Preferably, the cartridge further includes a flow path from the solvent chamber to each of the wells.

Preferably, the sample applicator includes a swab having a first and second swab heads for swabbing a surface to obtain a sample, the cartridge and sample applicator preferably being configured such that when the sample applicator is retained in the cartridge, the first swab head is retained in said preserving chamber to preserve a confirmatory version of said sample, and the second swab head is positioned in the solvent chamber to dissolve the second swab head to a substantially liquid mixture including said sample, and to permit the mixture to flow via the flow paths to the wells.

Preferably, the cartridge is positionable relative to the portable detection device to permit the portable detection device to detect the presence of at least one pathogen by sensing, and indicating the existence of, luminescence in one or more of said wells.

Preferably, the cartridge has a code element associated therewith, the code element including an indication of which pathogens are being tested for in each well. Preferably, the code element is a bar code.

Preferably, the detection device includes a code element reader for reading the code element. Preferably, the detection device includes a microprocessor for controlling functions of said detection device. Preferably, the detection device includes a display, operatively connected to the microprocessor, for displaying information regarding whether one or more pathogens have been detected. Preferably, the detection device is powered by a 9-volt battery. Preferably, the detection device includes an input device, operatively connected to the microprocessor, for programming the detection device. Preferably, the detection device includes a memory operatively connected to said microprocessor and said input device, said input device being configured to permit entry of test-related information for storage in the memory. Preferably, the detection device includes at least one software-programmable button operatively connected to the microprocessor, wherein the functions of said buttons can be programmed in said microprocessor. Preferably, the detection device includes a data transmission connection, operatively connected to the microprocessor, for transmitting data relating to pathogen detection from said detection device. Preferably, the detection device includes a code element reader positionable for reading the code element. Preferably, the detection device further includes an optical sensor module for detecting luminescence in said cartridge, the optical sensor module including a controller operatively connected to the microprocessor.

In another aspect, there is provided an optical sensor module for sensing luminescence resulting from the presence of a pathogen in one or more extraction chemicals, the module comprising a light sensor configured to emit a signal in response to incident light, and at least one amplification stage, operatively connected to the light sensor, for amplifying the signal to indicate a test result, the module including at least one noise filter for filtering noise from said signal. Preferably, the amplification stages are non-photosensitive. Preferably, the amplification stages comprise operational amplifiers. Preferably, each stage is operatively connected to a gain controller configured to independently control the gain of each stage. Preferably, the gain controller comprises a microcontroller programmed independently control the gain of each stage. Preferably, the module further includes dark current detector, the dark current detector being configured to determine that the test result is negative when only a background dark current signal is detected. Preferably, the dark current detector is a microcontroller. Preferably, the module includes a light maximizer (most preferably in the form of a lens) configured and positioned to maximize the effect of light from a well on said light sensor. Preferably, the module further includes a selector to select, one at a time, individual sets of extraction chemicals whose luminescence is to be sensed by said light sensor in testing for pathogens.

### BRIEF DESCRIPTION OF THE DRAWINGS

Reference will now be made, by way of example only, to drawings of the invention, which illustrate the preferred embodiment of the invention, and in which:

FIG. 1 is a plan view of a portion of the preferred detection device according to the present invention;

FIG. 2 is a side and plan view of the preferred test applicator kit according to the present invention;

FIG. 3 is a schematic diagram of the preferred wells and flow paths according to the present invention;

FIG. 4 is a schematic diagram of the preferred detection device according to the present invention; and

FIG. 5 is a schematic diagram of the preferred optical sensor module according to the present invention.

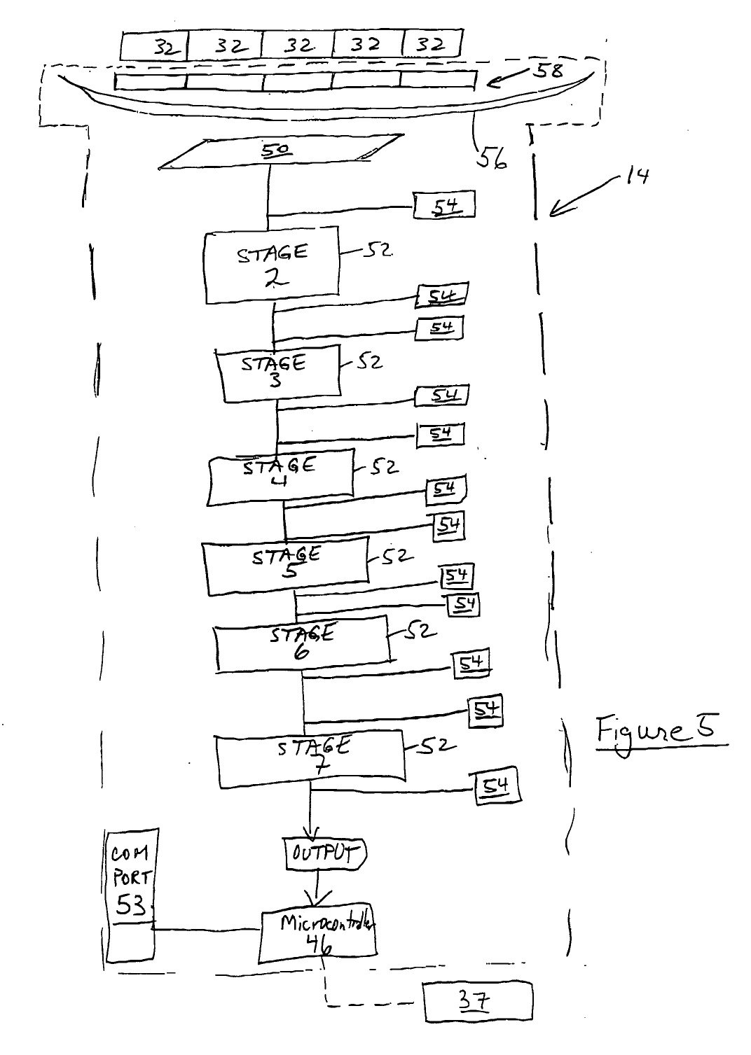

### DETAILED DESCRIPTION OF THE PREFERRED EMBODIMENTS

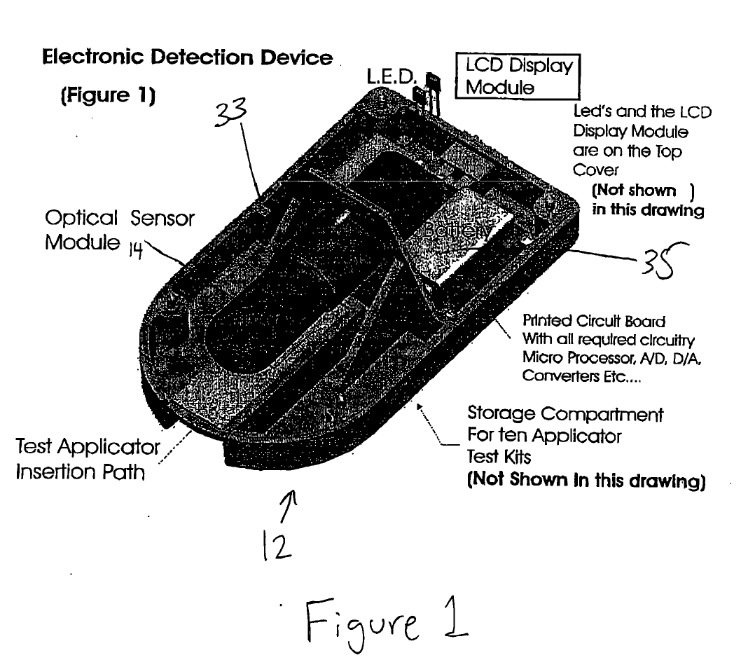

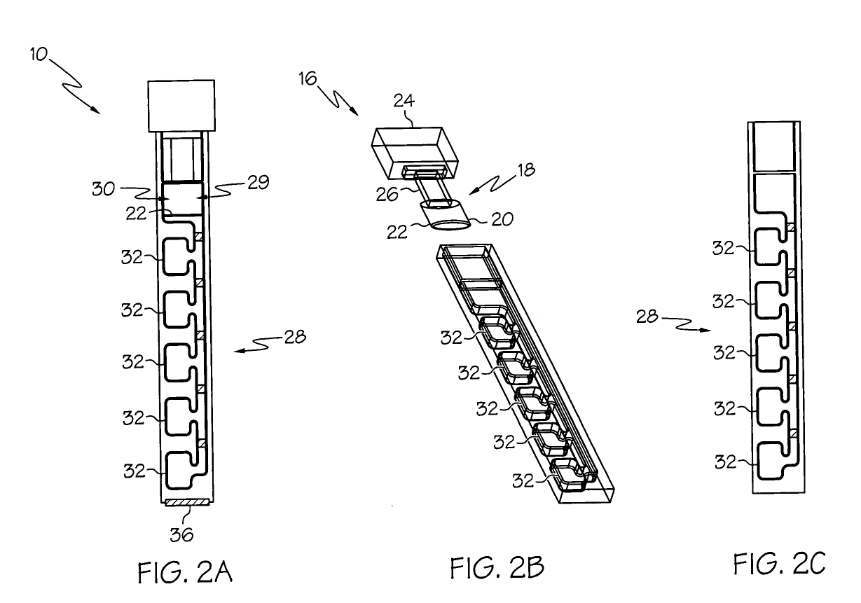

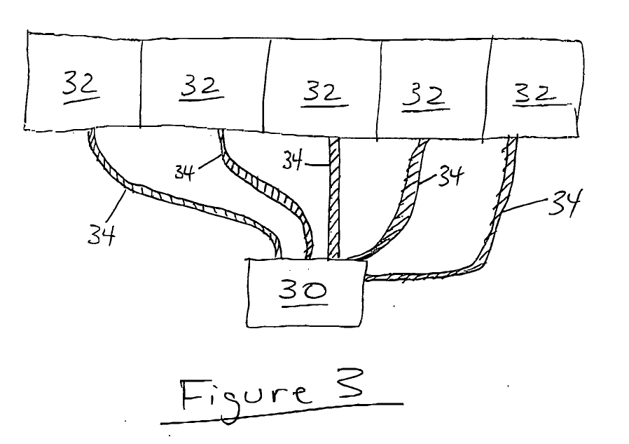

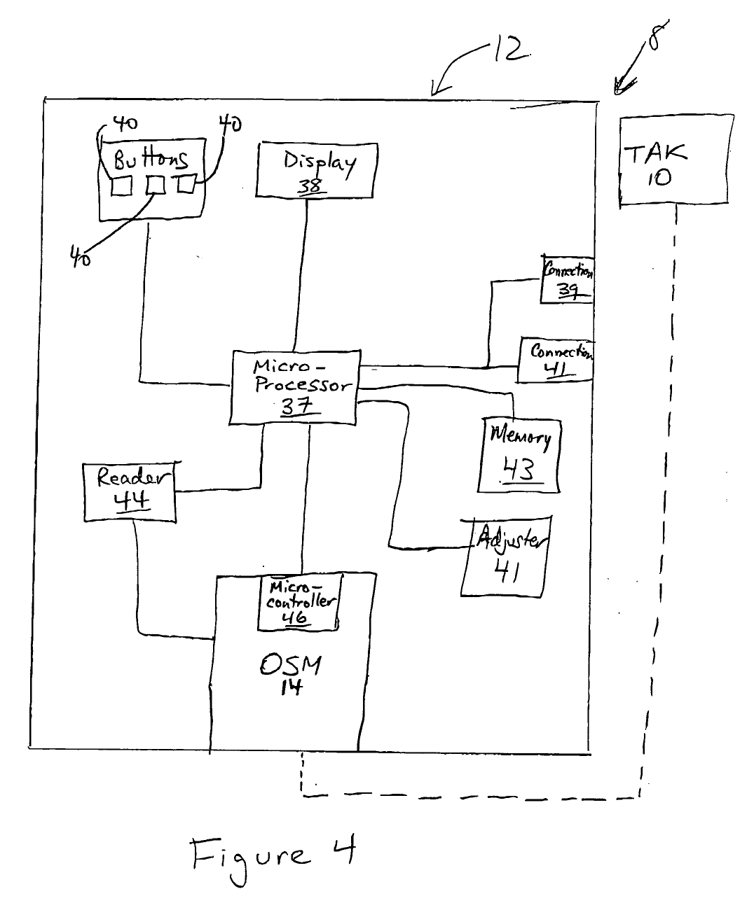

Referring now to FIGS. 1-4, the preferred contaminant detection apparatus 8 includes a test applicator kit (TAK) 10 and a detection device 12, with the detection device 12 including an optical sensor module (OSM) 14.

The preferred TAK 10 includes a dual-headed swab 18 (having heads 20 and 22) carried at the end of a sample applicator 16, the sample applicator 16 functioning as a sample collector. The swab 18 is used to swab surfaces or other areas from which a sample is desired. The sample applicator 16 further includes a handle 24 at one end of a body 26, with the dual-headed swab 18 being carried at an opposite end of the body 26.

The swab 18 is used to gather a sample for testing. Both heads 20 and 22 are brought into contact with the sample area. One of the heads 20 or 22 will, as will be described below, be inserted into a suspension fluid which preserves the sample. This preserved sample will act as a confirmatory sample, if it is desired to retest the sample at a later time.

The TAK 10 also includes a cartridge 28, configured to accept the sample applicator 16. Preferably, the cartridge 28 and sample applicator 16 are configured such that the sample applicator 16 can be inserted into the cartridge 28 with a snap fit, so that the sample applicator 16 is held in the cartridge 28 after the snap fit is engaged.

The cartridge 28 and applicator 16 are both sized, shaped and positioned so that when the snap fit is engaged, one of the heads (number 20) is preserved in a preserver, preferably in the form of a suspension fluid as mentioned above. The preserver is located in a preserving chamber 29. The other head (number 22) is at the same time positioned in a separate chamber 30 within the cartridge 28 containing a solvent. The solvent is selected to dissolve the head 22, so that the head 22, together with the sample on the head 22, dissolve into substantially liquid form.

The cartridge 28 preferably includes at least one well 32. More preferably, the cartridge 28 includes a plurality of wells 32, and most preferably, the cartridge 28 includes at least five wells 32. It will be appreciated by those skilled in the art that detection tests for pathogens are typically performed using extraction chemicals. These chemicals typically include, for example, alkaline phosphate and/or horseradish peroxide, a pathogen-specific antibody, and a pathogen-specific reactant chemical that causes light emission when the pathogen comes in contact with the extraction chemicals. The test is considered to have a positive result when light is emitted.

Thus, preferably, each well 32 contains, when the detection device 12 is in use, a pathogen-specific set of extraction chemicals. Most preferably, each well contains the chemicals necessary to test for a different pathogen. In this, way, multiple pathogens (most preferably, at least five) can be tested for using a single sample applicator 16 and cartridge 28 In one possible form of the cartridge 28, each well 32 comprises a single container for containing a pathogen-specific set of extraction chemicals. However, in another possible embodiment of the cartridge 28, each well 32 comprises a plurality of containers, with the plurality of containers preferably positioned one above the other. In this other possible embodiment, each extraction chemical from each pathogen-specific set of extraction chemicals is contained in a separate container. By contrast, in the single container embodiment, each set of pathogen-specific extraction chemicals is pre-mixed within the single container of which the well 32 is comprised. It will be appreciated that the multiple-container embodiment will preferably be used in situations where it is disadvantageous to mix the extraction chemicals from each set together before the test is performed.

In the multiple container embodiment, the cartridge 28 is most preferably configured so that, when the sample applicator 16 is snapped into the cartridge 28, the plurality of containers of which each well 32 is comprised are opened to one another. The result is that each extraction chemical is mixed together in each well 32 to form the corresponding pathogen-specific set of extraction chemicals.

Preferably, the cartridge 28 includes a plurality of flow paths 34 which place the chamber 30 in fluid communication with every well 32 in the cartridge 28. Most preferably, the flow paths 34 take the form of microchannels from the chamber 30 to each well 32. When the head 22 dissolves into liquid form, the dissolved liquid then travels to each well via the flow paths 34. The dissolved liquid contains the sample from the head 22. Therefore, the sample is carried via the flow paths 34 to each well 32, where the sample is introduced to each pathogen-specific set of extraction chemicals.

Preferably, the cartridge carries a code element in the form of one or more bar codes 36. The bar codes 36 contain within them information regarding the test including, preferably, the pathogens being tested for at each well 32, the date on which the TAK was manufactured, the expiry date of the TAK, and other relevant information.

As explained above, a positive test is preferably indicated by light being emitted from one or more of the wells 32 containing a pathogen-specific set of extraction chemicals. As will be more particularly described below, the detection device 12, and in particular the OSM 14, detects and indicates the presence of such light emissions in order to identify a positive test.

Preferably, the detection device 12 is battery-powered, most preferably by a 9-volt battery 35. The detection device 12 may also be powered by a rechargeable battery, AC power, or another power source. However, the use of a battery is preferred because this allows the apparatus 8 to be used more effectively in the field.

The device 12 preferably also includes a housing 33 for housing the components of the device 12, providing a support structure for them, and protecting same from the elements.

Preferably, the device 12 includes a microprocessor 37 for controlling the functions of the device 12 through software Thus, the processor 37 is operatively connected to, inter alia, the display 38, connections 39 and 41, memory 43, buttons 40, reader 44, microcontroller 46 and adjuster 41, which are described below.

Preferably, the device 12 also includes a display 38 for displaying information. Most preferably, the display 38 is a colour LCD display that is backlit. It will be appreciated by those skilled in the art that providing a colour display with backlighting facilitates the use of the device 12 in the field, including under darker field conditions where a different display would be more difficult to see. It is also preferred that this display comprise a touchscreen, so that information and programming can be inputted to the apparatus 8. It will be appreciated however, that other input devices besides a touchscreen are possible. What is desired is that the apparatus 8 include an input device to permit the apparatus 8 to be programmed.

Preferably, the device 12 includes a cellular phone connection 39 and/or a satellite telephone connection 41 to permit wireless communication from almost any location. It will be appreciated by those skilled in the art that, when testing for pathogens, the testing may be done in remote areas. Furthermore, it is often urgent that the test results be communicated as quickly as possible to the relevant authorities or other entities. For this reason, a connection of the sort just described is most preferred, as a data link is possible over such a connection. It will be appreciated that any communication connection over which data transmission is possible will serve this preferred function.

Preferably, the device 12 includes memory 43. The microprocessor preferably runs software that permits the user to enter all of the relevant information about the test being conducted (e.g. types of pathogens being tested for, date, location, client ID etc.). Preferably, the software that permits entry of relevant test information will also permit the downloading of said information to an external device, such as a PC. Preferably, the software will also permit this information, as well as information relating to the test results actually obtained, to be transmitted over the communication connection described above.

Preferably, the device 12 includes a control panel with software configurable buttons 40. The buttons 40 can serve a variety of functions depending on the preferences of the user. These include initiation of the test, control of the OSM 14 (as will be more particularly described below), the transmission and/or downloading of data, etc.

Preferably, the device 12 includes a built-in code element reader 44 in the form of a barcode scanner. The bar code scanner is configured to read the bar codes associated with the TAK 10 (and particularly the cartridge), which bar codes indicate what pathogens are being tested for. In addition, the OSM 14 of the device 12 will be programmable, so that the OSM 14 will automatically adjust to perform the test for the pathogen of interest. This adjustment may occur automatically using a preprogrammed microcontroller 46 associated with the OSM 14 in response to the reading of the code element. Alternatively, using the control panel, the microcontroller 46 can be programmed at the time of the test.

Preferably, the device 12 further includes a temperature adjuster 42 configured to adjust the temperature of the device 12. The most preferred form of adjuster is a thermoelectric heater/cooler operatively connected to the battery to draw power therefrom. It will be appreciated that proper testing for particular pathogens may require that the sample be held at a particular temperature. The preferred temperature adjuster is configured to adjust the temperature of the sample according to the requirements of the particular test.

It will be appreciated that the preferred TAK 10 and device 10 are configured so that, to perform the test, the user need only swab the surface, insert the applicator 16 into the cartridge 28, and insert the cartridge 28 into the OSM 14. This facilitates the use of the apparatus 8 in the field, because the addition of chemicals by robot, injector, or a technician is not required at test time.

The preferred OSM 14 will now be described. Preferably, the OSM 14 comprises a plurality of stages. The first stage preferably comprises a light sensor 50, most preferably in the form of a photo sensitive plate. It will be appreciated that the first stage may comprise any light sensor which emits a signal in response to incident light from a well 32.

The OSM 14 preferably includes a plurality of subsequent amplification stages 52. Most preferably, there are six stages 52. However, it will be appreciated that another number of stages 52 is possible. What is important is that the OSM 14 (preferably via the stages 50,52) emits a signal in response to light emission from a well 32, the signal being sufficiently strong to permit a positive test to be accurately recognized.

Preferably, each stage 52 comprises an operational amplifier. Preferably, each operational amplifier is operatively connected to the microcontroller 46. The microcontroller 46 is programmed to control the gain of each operational amplifier with precision.

Thus, preferably, each stage 52 has a corresponding gain which is independently adjustable i.e. adjustable independent from the gain at any other stage 52. This independent adjustability is advantageous, because it permits the microcontroller to exert tight control over the gain at each stage 52, as well as the overall gain of the OSM 14. This tight control in turn leads to more accurate test results. The reason for this is that this tight control prevents the gains of the op amps from floating independently away from their nominal values. Such floating could have the effect of skewing the test results. If the gain of one or more stages 52 were permitted to float (as sometimes undesirably happens with traditional photo multiplier tubes), an output signal that would, with the gains at their nominal values, have been above the threshold for a positive test result, might actually show up as being below the threshold for a positive test result, or vice versa. However, because the microcontroller 46 controls the gain of each stage 52 independently and precisely, the test results are more likely to be accurate, because the gains of each stage 52 are more likely to be at or close to their desired nominal values.

It will also be appreciated that the use of operational amplifiers as the stages 52 allows the stages 52 to be substantially more space sufficient than traditional photo multiplier tubes. Photo multiplier tubes typically use a photo sensitive plate for each amplification stage. These plates make the photo multiplier tube bulky, and inappropriate for use in the field. By contrast, the apparatus 8, which is preferably the size of a typical cellular phone, can be carried and used for effectively in the field. This is because the operational amplifiers are smaller than photosensitive plates and can also be implemented in the OSM 14 as surface mounted circuitry. Thus, the use of operational amplifiers as the stages 52 makes it possible for the apparatus 8 to be smaller, easier to carry, and easier to use in the field.

Preferably, each stage 52 will have associated therewith a noise filter 54 associated with the input to the stage 52, and a second noise filter 54 associated with the output from the stage. Preferably, the noise filter comprises circuitry designed to filter electromagnetic noise from the signal. It will be appreciated that the noise filters 54, being associated with both the input and output of each stage 52, can determine what portion of a received signal constitutes noise, because, given the tightly controlled gain of each stage 52, after the initial photo sensitive stage, the magnitude of the signal at each stage is predictable by the micro-controller 46 and noise filters 54.

Though it is preferred to have a noise filter associated with both the input and output of each stage 52, this degree of noise filtration is not required by the invention. The invention, for example, also comprehends a single noise filter 54 associated with each stage 52, or, alternatively, a one or more noise filters 54 which single handedly or collectively filter noise at all of the stages 52. The invention further comprehends one or more noise filters 54 that filter noise at one or more of the stages 52, but not necessarily all of the stages 52.

It will be appreciated that the use of one or more noise filters in association with the stages 52 increases the accuracy of the test result produced by the apparatus 8. For example, noise can lead to a false positive result if noise enters the signal and gets amplified. Alternatively, noise may swamp the signal produced by the light sensor 50, thus hiding a positive test result and falsely indicating a negative result.

At each stage 50, 52, a certain small amount of current, typically referred to as “background dark current” flows even when no light is being emitted and sent by the light sensor 50. This background dark current is the result of the voltages at the stages 50, 52. As a result, the OSM 14 would tend to produce a non-zero signal because of the background dark current even when no light is being emitted Therefore, preferably, the micro-controller 46 is programmed to measure the signal associated with the background dark current, and to interpret that background dark current signal as a zero signal. It will be appreciated that the micro-controller 46 thus improves the accuracy of the test results produced by the apparatus 8. Since the background dark current signal is present when there is a zero light emission, the test is most accurate when the micro-controller 46 subtracts the background current signal from the actual signal outputted from the stages 52 to determine the actual signal produced by the light sensor 50 and stages 52.

Preferably, the micro-controller is programmed/configured to compensate for changes in the temperature of the OSM 14. As will be appreciated by those skilled in the art, when temperatures are higher, more current flows. Thus, the signal produced by the OSM 14 in response to a light emission in the well 32 depends on the temperature of the OSM 14 and the circuitry contained therein. Thus, the micro-controller is preferably configured adjust the gains of the stages 52 according to the temperature of the OSM 14 and apparatus 8. The preferred result is that, if the same test on the same sample is performed under different temperature conditions, the results will be consistent because of the temperature compensation.

It will be appreciated that the micro-controller 46, acting as a temperature compensator, provides significant advantages for the apparatus 8. Specifically, the apparatus 8 can be more effectively used in the field, where temperatures can vary widely. By contrast in a laboratory, the temperature is more carefully controlled.

Preferably, the OSM 14 includes an automatic gain adjustor in the form of the micro-controller 46. Specifically, the micro-controller 46 is preferably configured to adjust the gains of the stages 52 automatically, up to predetermined maximums, when a negative test result is initially indicated. Thus, if a negative test result is initially detected, the micro-controller 46 will preferably increase the gain of the stages 52 incrementally and again check the test result to see if it is negative. The micro-controller 46 will continue to raise the gain of the stages 52 up to the predetermined maximums, or until a positive test result is obtained, whichever comes first.

It will be appreciated, therefore, that the micro-controller 46 acts as a test sensitivity adjustor, acting automatically to increase the sensitivity of the test up to a predetermined maximum. This allows the OSM 14 to adapt to different test conditions, and to determine test results with greater certainty.

Preferably, the OSM 14 is a self-contained unit which includes a serial communications port 53, or other communications port for communicating with the micro-processor. It will be appreciated that, in this way, the data from the OSM 14 can be communicated quickly and easily to the micro-processor, which in turn can process the data, display related information to the user, and transmit related information to other locations through the communication connections described above.

It will be appreciated that, though the OSM 14 forms part of the apparatus 8, it can also be used in association with separate devices not comprehended by the apparatus 8 and the device 12. Specifically, the micro-controller 46 and communications port 53 permit the OSM to be used in association with other devices or machines which can receive and process the data outputted by the OSM 14. Thus, the OSM 14 can be installed as a separate component in other machines or devices, and sold separately for use in association with such other machines and devices.

Preferably, the OSM 14 further includes a lens 56. Preferably, the device 12 and OSM 14 are configured so that the lens 56 is positioned so as to focus light emitted from the wells 32 onto the light sensor 50. It will be appreciated, therefore, that the lens 56 functions as light maximizer. By focusing the light emitted from the wells 32 on to the light sensor 50, the lens 56 maximizes the effect of the light by focusing the light energy directly onto the light sensor 50.

The OSM 14 preferably further includes a selector, preferably in a form of a shutter array 58. Preferably, there will be one shutter in the shutter array for each well 32. The selector functions to block all of the wells 32 from emitting light onto the lens 56 and sensor 50, except for one well 32. In other words, the selector functions to permit one well 32 at a time to be tested. Thus, in the preferred embodiment where there five wells 52, the selector will expose the first well 32 to the lens 56, but bock all of the others until the test result from the first well 32 is obtained. The selector will then block the first well 32, and expose the second well 32, and repeat the process. This process is then repeated for each of the third, fourth and fifth wells.

Thus, the selector, operatively connected to the micro-controller 46, selects which well is being tested, and therefore, which pathogen is being tested for. Once the test associated with a particular well 32 is complete, and the test results obtained, the next well 32 is exposed, the test completed, and the test results obtained.

It will be appreciated that the invention comprehends that there be no selector, but instead that the OSM be configured so that the light from each well 32 is emitted onto a separate light sensor 50. However, it has been found that it is more cost and space efficient to have the selector which functions to select one well 32 at a time for obtaining a test result.

The above disclosure is intended to be illustrative and not exhaustive. The description will suggest many variations and alternatives to one of ordinary skill in the art. All these alternatives and variations are intended to be included within the scope of the claims, in which the terms “comprise” and “include” mean “including, but not limited to.”

Further, the particular features presented in the disclosure and the claims can be combined with each other in other manners within the scope of the invention such that the invention should be recognized as also specifically directed to other embodiments having other possible combinations of the features of the claims

1. A contaminant detection apparatus comprising:
a powered portable detection device for detecting the presence of at least one pathogen in a sample;
a test applicator kit comprising a sample applicator and a cartridge configured to receive and retain the sample applicator, the cartridge including a solvent chamber for holding a solvent, a preserving chamber for holding a preserver, and at least one well for holding at least one pathogen-specific set of extraction chemicals, the cartridge further including a flow path from the solvent chamber to each of the wells;
the sample applicator including a swab having a first and second swab heads for swabbing a surface to obtain a sample, the cartridge and sample applicator being configured such that when the sample applicator is retained in the cartridge, the first swab head is retained in said preserving chamber to preserve a confirmatory version of said sample, and the second swab head is positioned in the solvent chamber to dissolve the second swab head to a substantially liquid mixture including said sample, and permit the mixture to flow via the flow paths to the wells;
the cartridge being positionable relative to the portable detection device to permit the portable detection device to detect the presence of at least one pathogen by sensing, and indicating the existence of, luminescence in one or more of said wells.

2. The apparatus of claim 1, wherein the cartridge has a code element associated therewith, the code element including an indication of which pathogens are being tested for in each well.

3. The apparatus of claim 2, wherein the code element is a bar code.

4. The apparatus of claim 2, wherein the detection device includes a code element reader for reading the code element.

5. The apparatus of claim 1, wherein the detection device includes a microprocessor for controlling functions of said detection device.

6. The apparatus of claim 5, wherein the detection device includes a display, operatively connected to the microprocessor, for displaying information regarding whether one or more pathogens have been detected.

7. The apparatus of claim 5, wherein the detection device is powered by a 9-volt battery.

8. The apparatus of claim 6, wherein said detection device includes an input device, operatively connected to the microprocessor, for programming the detection device.

9. The apparatus of claim 8, wherein said detection device includes a memory operatively connected to said microprocessor and said input device, said input device being configured to permit entry of test-related information for storage in the memory.

10. The apparatus of claim 6, wherein the detection device includes at least one software-programmable button operatively connected to the microprocessor, wherein the functions of said buttons can be programmed in said microprocessor.

11. The apparatus of claim 6, wherein the detection device includes a data transmission connection, operatively connected to the microprocessor, for transmitting data relating to pathogen detection from said detection device.

12. The apparatus of claim 6, wherein the detection device further includes an optical sensor module for detecting luminescence in said cartridge, the optical sensor module including a controller operatively connected to the microprocessor.

13. An optical sensor module for sensing luminescence resulting from the presence of a pathogen in one or more extraction chemicals, the module comprising a light sensor configured to emit a signal in response to incident light, and at least one amplification stage, operatively connected to the light sensor, for amplifying the signal to indicate a test result, the module including at least one noise filter for filtering noise from said signal.

14. The module of claim 13, wherein the amplification stages comprise operational amplifiers.

15. The module of claim 13, wherein each stage is operatively connected to a gain controller configured to independently control the gain of each stage.

16. The module of claim 15, wherein the gain controller comprises a microcontroller programmed independently control the gain of each stage.

17. The module of claim 13, the module further including a dark current detector, the dark current detector being configured to determine that the test result is negative when only a background dark current signal is detected.

18. The module of claim 17, wherein the dark current detector is a microcontroller.

19. The module of claim 13, wherein the module includes a light maximizer configured and positioned to maximize the effect of light from a well on said light sensor.

20. The module of claim 13, wherein the module further includes a selector to select, one at a time, individual sets of extraction chemicals whose luminescence is to be sensed by said light sensor in testing for pathogens.
